(19) United States
(12) Patent Application Publication
(10) Pub. No.: US 2012/0238874 A1
(43) Pub. Date: Sep. 20, 2012

(54) ULTRASOUND DIAGNOSTIC APPARATUS AND ULTRASOUND IMAGE PRODUCING METHOD

(75) Inventor: Yukiya MIYACHI, Ashigara-kami-gun (JP)

(73) Assignee: FUJIFILM CORPORATION, Tokyo (JP)

(21) Appl. No.: 13/397,375

(22) Filed: Feb. 15, 2012

(30) Foreign Application Priority Data
Mar. 18, 2011 (JP) 2011-060316

(51) Int. Cl. A61B 8/14

(52) U.S. Cl. 600/443

(57) ABSTRACT

An ultrasound diagnostic apparatus includes a region-of-interest setting unit for setting a region of interest in the B mode image, an abdominal wall detector for detecting an abdominal wall of the subject in the B mode image, a controller for controlling a transmission circuit and a reception circuit to transmit a ultrasonic beam emitted from the transducer array and so steered as to enter the abdominal wall detected by the abdominal wall detector vertically with forming transmission focuses respectively at a plurality of points set in and near the region of interest to obtain reception data for measuring a sound speed, and a sound speed calculator for calculating local sound speeds in the region of interest based on the obtained reception data for measuring a sound speed.

### BACKGROUND OF THE INVENTION

The present invention relates to an ultrasound diagnostic apparatus and an ultrasound image producing method and particularly to an ultrasound diagnostic apparatus for both producing a B mode image and measuring a sound speed.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits an ultrasonic beam toward the inside of a subject's body, receives ultrasonic echoes from the subject, and the apparatus body electrically processes the reception signals to produce an ultrasound image.

In recent years, sound speeds in a region under examination are measured to achieve a more accurate diagnosis of the region inside the subject's body.

JP 2010-99452 A, for example, proposes an ultrasound diagnostic apparatus whereby a plurality of lattice points are set around a site under examination and an ultrasonic beam is transmitted to and received from the lattice points to obtain reception data, based on which local sound speeds are calculated.

### SUMMARY OF THE INVENTION

JP 2010-99452 A describes a device having an ultrasound probe that transmits and receives an ultrasonic beam to and from the inside of a subject's body to obtain local sound speeds at a site under examination, thereby enabling display of a B mode image with, for example, the local sound speeds superimposed over it. Further, producing a sound speed map representing a distribution of local sound speeds at respective points in a given region and displaying it together with the B mode image effectively support diagnosis of a site under examination.

However, because the sound speed at points near the abdominal wall covering organs, for examples, is different from that in other points because of the existence of fat among other causes, there arises a problem that as an ultrasonic beam emitted from the ultrasound probe passes through the abdominal wall, the ultrasonic beam may be refracted depending on the angle of incidence with respect to the abdominal wall, possibly making an accurate measuring impossible.

An object of the present invention is to eliminate such problems associated with the prior art and provide an ultrasound diagnostic apparatus and an ultrasound image producing method capable of accurate measuring a sound speed by reducing the effects of refraction of a ultrasonic beam caused by an abdominal wall.

An ultrasound diagnostic apparatus according to the present invention comprises:

a transducer array;

a transmission circuit for transmitting an ultrasonic beam from the transducer array toward a subject;

a reception circuit for processing reception signals outputted from the transducer array having received ultrasonic echoes from the subject to produce reception data;

an image producer for producing a B mode image based on the reception data obtained by the reception circuit;

a region-of-interest setting unit for setting a region of interest in the B mode image produced by the image producer;

an abdominal wall detector for detecting an abdominal wall of the subject in the B mode image produced by the image producer;

a controller for controlling the transmission circuit and the reception circuit to transmit a ultrasonic beam emitted from the transducer array and so steered as to enter the abdominal wall detected by the abdominal wall detector vertically with forming transmission focuses respectively at a plurality of points set in and near the region of interest to obtain reception data for measuring a sound speed; and

a sound speed calculator for calculating local sound speeds in the region of interest based on the obtained reception data for measuring a sound speed.

An ultrasound image producing method according to the present invention comprises the steps of:

transmitting an ultrasonic beam from a transducer array toward a subject;

producing reception data based on reception signals outputted from the transducer array having received ultrasonic echoes from the subject;

producing a B mode image based on the obtained reception data;

setting a region of interest in the produced B mode image;

detecting an abdominal wall of the subject in the B mode image;

setting a plurality of points in and near the region of interest;

transmitting and receiving ultrasonic beams emitted from the transducer array and so steered as to enter the abdominal wall vertically with forming transmission focuses at the points to obtain reception data for measuring a sound speed; and

calculating local sound speeds in the region of interest based on the obtained reception data for measuring a sound speed.

### BRIEF DESCRIPTION OF THE DRAWINGS

FIG. 1 is a block diagram illustrating a configuration of an ultrasound diagnostic apparatus according to Embodiment 1 of the invention.

FIGS. 2A and 2B schematically illustrate a principle of sound speed calculation in Embodiment 1.

FIG. 3 illustrates an ultrasonic beam transmitted toward a region of interest in Embodiment 1.

FIG. 4 illustrates lattice points set in Embodiment 1.

FIG. 5 illustrates ultrasonic beams transmitted toward a region of interest according to a variation of Embodiment 1.

FIG. 6 illustrates ultrasonic beams transmitted toward a plurality of regions of interest in Embodiment 2.

FIG. 7 illustrates how a local sound speed in a region of interest is calculated by interpolation in Embodiment 2.

### DETAILED DESCRIPTION OF THE INVENTION

Embodiments of the present invention will now be described below based on the appended drawings.

### Embodiment 1

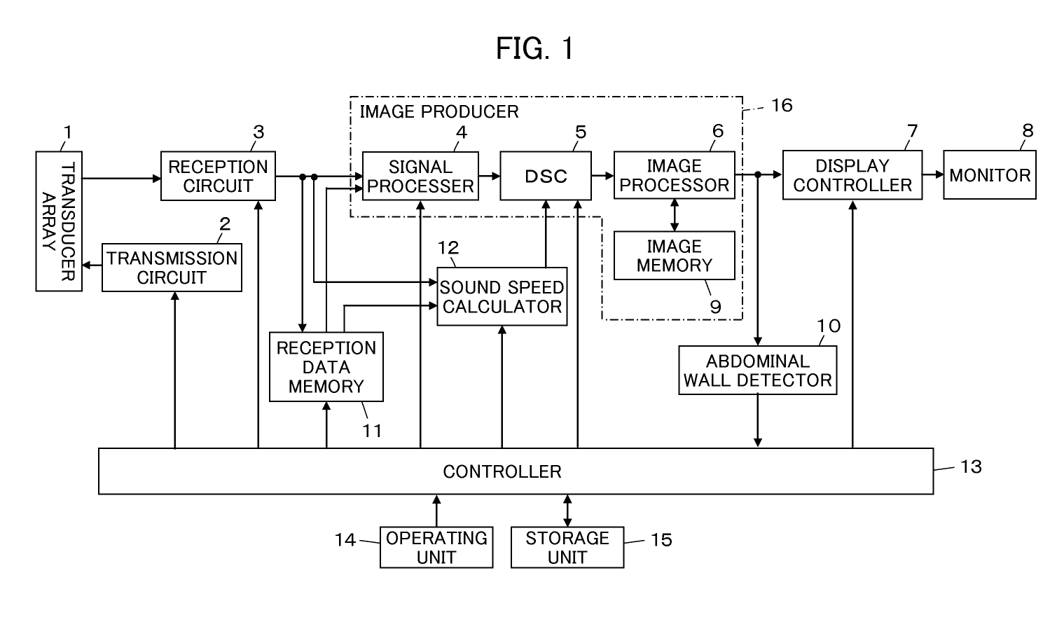

FIG. 1 illustrates a configuration of an ultrasound diagnostic apparatus according to Embodiment 1 of the invention. The ultrasound diagnostic apparatus comprises a transducer array 1, which is connected to a transmission circuit 2 and a reception circuit 3. The reception circuit 3 is connected in sequence to a signal processor 4, a DSC (Digital Scan Converter) 5, an image processor 6, a display controller 7, and a monitor 8. The image processor 6 is connected to an image memory 9 and an abdominal wall detector 10. The reception circuit 3 is also connected to a reception data memory 11 and a sound speed calculator 12.

The signal processor 4, the DSC 5, the display controller 7, the abdominal wall detector 10, the reception data memory 11, and the sound speed calculator 12 are connected to a controller 13. The controller 13 is also connected to an operating unit 14 and a storage unit 15.

The transducer array 1 comprises a plurality of ultrasound transducers arranged one-dimensionally or two-dimensionally. These ultrasound transducers each transmit ultrasonic waves according to actuation signals supplied from the transmission circuit 2 and receive ultrasonic echoes from the subject to output reception signals. Each of the ultrasound transducers comprises a vibrator composed of a piezoelectric body and electrodes each provided on both ends of the piezoelectric body. The piezoelectric body is composed of, for example, a piezoelectric ceramic represented by a PZT (titanate zirconate lead), a polymeric piezoelectric device represented by PVDF (polyvinylidene flouride), or a piezoelectric monochristal represented by PMN-PT (lead magnesium niobate lead titanate solid solution).

When the electrodes of each of the vibrators are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce an electric signal, which is then outputted as reception signal for the ultrasonic waves.

The transmission circuit 2 includes, for example, a plurality of pulsars and adjusts the delay amounts for actuation signals based on a transmission delay pattern selected according to an instruction signal transmitted from the transmission controller 13 so that the ultrasonic waves transmitted from a plurality of ultrasound transducers of the transducer array 1 form an ultrasonic beam and supplies the ultrasound transducers with delay-adjusted actuation signals.

The reception circuit 3 amplifies and A/D converts the reception signals transmitted from the respective ultrasonic transducers of the transducer array 1 to produce reception data.

The signal processor 4 performs reception focusing processing by providing the reception signals produced by the reception circuit 3 with respective delays according to a sound speed or a sound speed distribution that is set based on a reception delay pattern selected according to the control signal from the controller 13, followed by addition, to produce a sound ray signal where the ultrasonic echoes are well focused and, upon correcting the attenuation according to the distance depending on the depth at which the ultrasonic waves are reflected, performs envelope detection processing to produce a B mode image signal, which is tomographic image information on a tissue inside the subject's body.

The DSC 5 converts the B mode image signal produced by the signal processor 4 into an image signal compatible with an ordinary television signal scanning mode (raster conversion).

The image processor 6 performs various processing required including gradation processing on the B mode image signal entered from the DSC 5 before outputting the B mode image signal to the display controller 7 or storing the B mode image signal in the image memory 9.

The signal processor 4, the DSC 5, the image processor 6, and the image memory 9 constitute an image producer 16.

The display controller 7 causes the monitor 8 to display an ultrasound diagnostic image according to the B mode image signal having undergone image processing by the image processor 6.

The monitor 8 includes a display device such as an LCD, for example, and displays an ultrasound diagnostic image under the control of the display controller 7.

The abdominal wall detector 10 detects a subject's abdominal wall P in the B mode image according to the B mode image signal image-processed by the image processor 6 and detects the shape of the abdominal wall P.

The reception data memory 11 stores the reception data outputted from the reception circuit 3 sequentially by channel. The reception data memory 11 stores information on a frame rate entered from the controller 13 in association with the above reception data. Such information includes, for example, the depth of a position at which the ultrasonic wave is reflected, the density of scan lines, and a parameter representing the range of the visual field.

Under the control by the controller 13, the sound speed calculator 12 calculates the local sound speeds in a tissue inside the subject's body under examination based on the reception data stored in the reception data memory 11.

The controller 13 controls the components in the ultrasound diagnostic apparatus according to the instruction entered by the operator using the operating unit 14.

The operating unit 14, provided for the operator to perform input operations, constitutes a region-of-interest setting unit and may be composed of, for example, a keyboard, a mouse, a track ball, and/or a touch panel.

The storage unit 15 stores, for example, an operation program and may be constituted by, for example, a recording medium such as a hard disk, a flexible disk, an MO, an MT, a RAM, a CD-ROM, a DVD-ROM, an SD card, a CF card, or a USB memory, a server, or the like.

The signal processor 4, the DSC 5, the image processor 6, the display controller 7, and the sound speed calculator 12 are each constituted by a CPU and an operation program for causing the CPU to perform various kinds of processing, but they may be each constituted by a digital circuit.

The operator may select one of the following three display modes using the operating unit 14. They are: a mode for displaying the B mode image alone, a mode for displaying the B mode image, with the local sound speeds in the region of interest superimposed over the B mode image, and a mode for displaying the B mode image and the local sound speeds in juxtaposition.

To display the B mode image, a plurality of ultrasound transducers of the transducer array 1 first transmit ultrasonic waves according to the actuation signals supplied from the transmission circuit 2, and the ultrasound transducers having received ultrasonic echoes from the subject output the reception signals to the reception circuit 3, which produces the reception data. The signal processor 4, having received the reception data, produces the B mode image signal, the DSC 5 performs raster conversion of the B mode image signal, and the image processor 6 performs various image processing on the B mode image signal, whereupon, based on this B-mode image signal, the display controller 7 causes the monitor 8 to display the ultrasound diagnostic image.

The local sound speed may be calculated by, for example, a method described in JP 2010-99452 A filed by the Applicant of the present application.

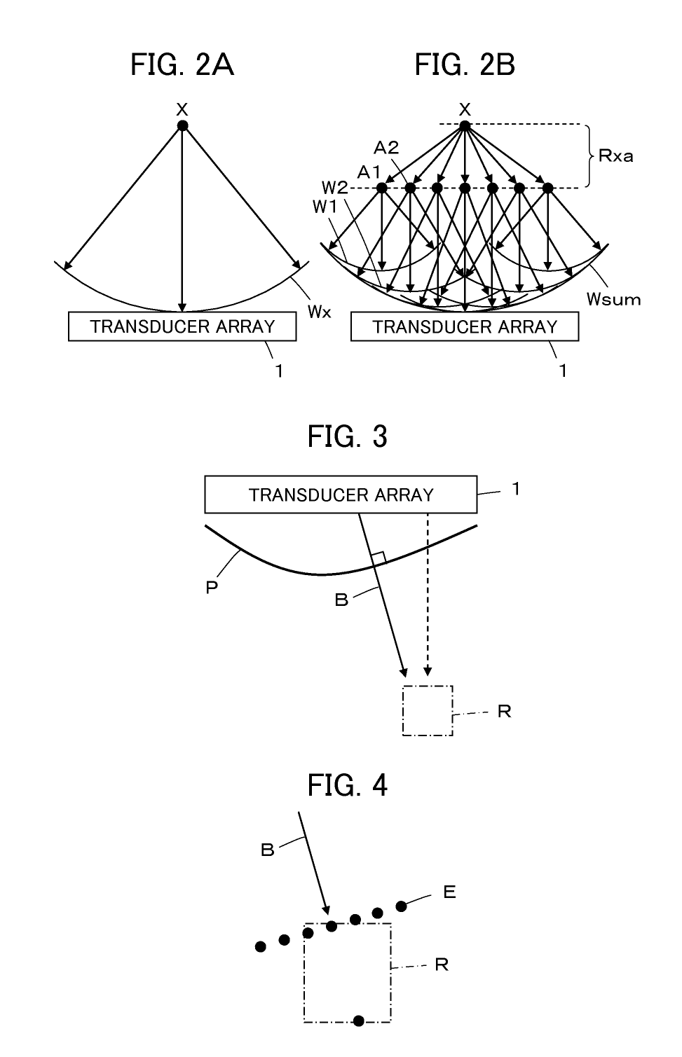

This method obtains the local sound speed at a lattice point X according to the Huygens principle. Suppose now that, on transmission of ultrasonic waves to the inside of a subject, a reception wave Wx reaches the transducer array 1 from the lattice point X, a reflection point in the subject, as illustrated in FIG. 2A, and that a plurality of lattice points A1, A2, . . . are arranged at equal intervals in positions shallower than the lattice point X, i.e., closer to the transducer array 1, as illustrated in FIG. 2B. Then, the local sound speed at the lattice point X is obtained according to the Huygens principle whereby a synthesized wave Wsum produced by combining reception waves W1, W2, . . . transmitted from the lattice points A1, A2, . . . having received a reception signal from the lattice point X coincides with the reception wave Wx from the lattice point X.

First, optimum sound speeds for all the lattice points X, A1, A2, . . . are obtained. The optimum sound speed herein means a sound speed allowing a highest image contrast and sharpness to be obtained as a set sound speed is varied after performing focus calculation for the lattice points based on the set sound speed and imaging to produce an ultrasound image. The optimum sound speed may be judged based on, for example, the image contrast, spatial frequency in the scan direction, and dispersion as described in JP 08-317926 A.

Next, the optimum sound speed for the lattice point X is used to calculate the waveform of an imaginary reception waves Wx emitted from the lattice point X.

Further, a hypothetical local sound speed V at the lattice point X is changed to various values to calculate the imaginary synthesized wave Wsum of the reception waves W1, W2, . . . from the lattice points A1, A2, . . . Suppose that, at this time, the sound speed is consistent in a region Rxa between the lattice point X and the lattice points A1, A2, . . . and is equivalent to the local sound speed V at the lattice point X. The times in which the ultrasonic wave propagating from the lattice point X reaches the lattice points A1, A2, . . . are XA1/V, XA2/V, . . . , respectively, where XA1, XA2, . . . are the distances between the lattice point X and the lattice points A1, A2, . . . Combining the reflected waves emitted from the lattice points A1, A2, . . . with respective delays corresponding to the times XA1/V, XA2/V, . . . yields the imaginary synthesized wave Wsum.

Next, the respective differences between a plurality of the imaginary synthesized waves Wsum calculated by changing the hypothetical local sound speed V at the lattice point X to various values and the imaginary reception waves Wx from the lattice point X are calculated to determine the hypothetical local sound speed V at which the difference becomes a minimum as the local sound speed. The difference between the imaginary synthesized waves Wsum and the imaginary reception waves Wx from the lattice point X may be calculated by any of appropriate methods including a method using the cross-correlation, a method using phase matching addition by multiplying the reception waves Wx by a delay obtained from the synthesized wave Wsum, and a method using phase matching addition by multiplying the synthesized wave Wsum by a delay obtained from the reception waves Wx.

Thus, the local sound speeds inside a subject can be accurately calculated based on the reception data produced by the reception circuit 3. The sound speed map representing a distribution of the local sound speeds in a set region of interest may be likewise produced.

Next, the operation of Embodiment 1 will be described.

First, according to the actuation signal from the transmission circuit 2, a plurality of ultrasound transducers of the transducer array 1 transmit an ultrasonic beam, and the ultrasound transducers having received ultrasound echoes from a subject output reception signals to the reception circuit 3 to produce reception data, whereupon the display controller 7 causes the monitor 8 to display the B mode image based on the B mode image signal produced by the image producer 16.

Now, the operator operates the operating unit 14 to set a region of interest R in the B mode image displayed on the monitor 8, whereupon the abdominal wall detector 10 detects the shape of the subject's abdominal wall P located between the transducer array 1 and the region of interest R. The shape of the abdominal wall P is transmitted to the controller 13 and, as illustrated in FIG. 3, the controller 13 sets a control signal for a transmission delay pattern whereby the ultrasonic beam B transmitted from the transducer array toward the region of interest R is so steered as to enter the abdominal wall P detected by the abdominal wall detector 10 at a steering angle that is substantially perpendicular to the abdominal wall P. As the actuation signal is supplied from the transmission circuit 2 to the ultrasound transducers according to such transmission pattern, the ultrasonic beam can be steered so as to enter the abdominal wall P substantially at right angles instead of being transmitted in a direction perpendicular to the transducer array 1. Further, the controller 13 sets a plurality of lattice points near the region of interest R so as to sandwich the region of interest R. As illustrated in FIG. 4, for example, a plurality of lattice points E may be set so as to be perpendicular to the steered ultrasonic beam B.

Next, the transmission circuit 2 and the reception circuit 3 are controlled by the controller 13 according to the thus set control signal, and ultrasonic beams B emitted from the transducer array 1 to enter the abdominal wall P substantially at right angles and form transmission focuses at their respective lattice points E are received in sequence. The thus transmitted ultrasonic beams B, entering the abdominal wall P substantially at right angles, can pass through the abdominal wall P, virtually without being affected by refraction through the abdominal wall P, to form transmission focuses at their respective lattice points.

Subsequently, the reception data for measuring the sound speed produced by the reception circuit 3 each time the ultrasonic beam is received are sequentially stored in the reception data memory 11. When the reception data for measuring the sound speed have been stored in the reception data memory 11 for all the lattice points E that are set near the region of interest R, the sound ray calculator 12 uses the reception data for measuring the sound speed stored in the reception data memory 11 to calculate the local sound speeds in the region of interest R on the assumption that the sound speed is consistent inside the region of interest R that is sandwiched by the lattice points E having different depths.

Thus, the ultrasonic beam can be allowed to enter the subject's abdominal wall P substantially at right angles by adjusting the steering angle of the transmitted ultrasonic beam according to the control signal, so that the effects of refraction of the ultrasonic beam produced by the abdominal wall P can be reduced, achieving an accurate sound speed measuring.

The accuracy of the sound speed measuring may also be enhanced by transmitting the ultrasonic beam B a plurality of times while slightly changing the steering angle of the ultrasonic beam B that is so transmitted from the transducer array 1 as to enter the abdominal wall P substantially at right angles.

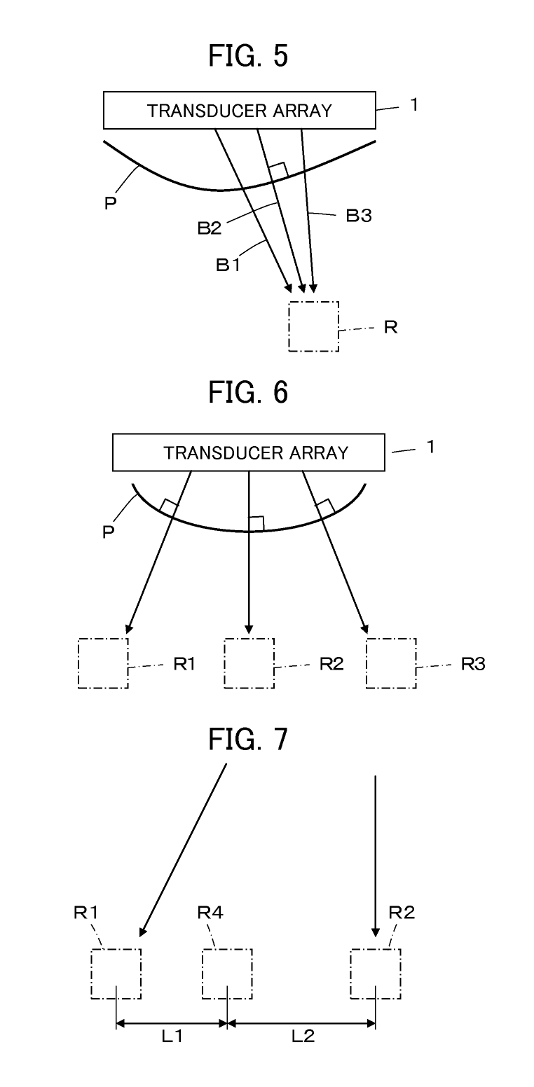

Upon detection of the shape of the subject's abdominal wall P by the abdominal wall detector 10, the controller 13 obtains a steering angle of the ultrasonic beam B allowing the ultrasonic beam B to enter the abdominal wall P substantially at right angles while setting a control signal for controlling the transmission delay pattern for transmitting ultrasonic beams B1, B2, and B3 having slightly different steering angles with respect to the same region of interest R as illustrated in FIG. 5. Based on the thus set control signal, the controller 13 controls the transmission circuit 2 and the reception circuit 3 to transmit and receive the ultrasonic beams B1, B2, and B3 through the transducer array 1 to and from the same region of interest R to obtain a plurality of reception data. Now, the ultrasonic beam B2 allowed to enter the abdominal wall P substantially at right angles yields reception data where the wave front is only slightly disturbed because the effects of refraction caused by the abdominal wall P is small, whereas the ultrasonic beams B1 and B3 not steered to enter the abdominal wall P at right angles yield reception data where the wave front is disturbed by the effects of refraction caused by the abdominal wall P. Therefore, the sound speed calculator 12 uses the reception data having a wave front least disturbed among a plurality of reception data obtained from the ultrasonic beams B1, B2, and B3 as reception data for calculating the sound speed to calculate the local sound speeds inside the region of interest R.

Thus, using the reception data least affected by refraction of the ultrasonic beam caused by the abdominal wall P among a plurality of reception data obtained by transmitting and receiving a plurality of ultrasonic beams each having slightly different steering angles with respect to the region of interest R as reception data for measuring the sound speed enables an accurate sound speed measuring.

### Embodiment 2

A plurality of regions of interest R may also be provided in the B mode image, so that the sound speed calculator 12 calculates the local sound speeds in the respective regions of interest to produce the sound speed map for a region containing these regions of interest R.

As illustrated in FIG. 6, for example, with three regions of interest R1, R2, ad R3 set in the B mode image, ultrasonic beams each steered in three directions allowing them to enter the abdominal wall P located between the transducer array 1 and the respective regions of interest R1, R2, ad R3 substantially at right angles are transmitted and received likewise as according to Embodiment 1.

Based on the reception data for measuring the sound speed thus received, the sound speed calculator 12 calculates the respective local sound speeds in the regions of interest R1, R2, ad R3. Further, the sound speed calculator 12 calculates the local sound speeds in the positions between the regions of interest R1, R2, and R3 by interpolation using the calculated respective local sound speeds.

The local sound speed in a region of interest R4 between the regions of interest R1 and R2, for example, may be interpolated by averaging using the local sound speeds in the regions of interest R1 and R2 and considering a distance L1 between the regions of interest R1 and R4 and a distance L2 between the regions of interest R2 and R4 as illustrated in FIG. 7. The interpolation is repeated to produce a sound speed map of a region containing the regions of interest R1, R2, and R3.

The data on the sound speed map obtained by the sound speed calculator 12 are allowed to undergo raster conversion by the DSC 5 and various image processing by the image processor 6 before being transmitted to the display controller 7. Then, according to the display mode entered from the operating unit 14 by the operator, the B mode image is displayed, with the sound speed map superimposed over it, on the monitor 8, or the B mode image and the sound speed map are displayed in juxtaposition on the monitor 8.

Thus, an accurate sound speed map can be produced based on the reception data where the effects of refraction of the ultrasonic beam caused by the abdominal wall P are reduced. Further, a distortion-free sound speed map can be produced by interpolating a local sound speed in a region located between adjacent regions of interest using local sound speeds in these regions of interest obtained by transmitting and receiving the ultrasonic beam.

What is claimed is:

1. An ultrasound diagnostic apparatus comprising:
a transducer array;
a transmission circuit for transmitting an ultrasonic beam from the transducer array toward a subject;
a reception circuit for processing reception signals outputted from the transducer array having received ultrasonic echoes from the subject to produce reception data;
an image producer for producing a B mode image based on the reception data obtained by the reception circuit;
a region-of-interest setting unit for setting a region of interest in the B mode image produced by the image producer;
an abdominal wall detector for detecting an abdominal wall of the subject in the B mode image produced by the image producer;
a controller for controlling the transmission circuit and the reception circuit to transmit a ultrasonic beam emitted from the transducer array and so steered as to enter the abdominal wall detected by the abdominal wall detector vertically with forming transmission focuses respectively at a plurality of points set in and near the region of interest to obtain reception data for measuring a sound speed; and
a sound speed calculator for calculating local sound speeds in the region of interest based on the obtained reception data for measuring a sound speed.

2. The ultrasound diagnostic apparatus according to claim 1, wherein the controller sets the points so as to be located on a line vertical to the steered ultrasonic beam.

3. The ultrasound diagnostic apparatus according to claim 1,
wherein the controller controls the transmission circuit and the reception circuit to obtain a plurality of reception data for measuring a sound speed by transmitting and receiving a plurality of ultrasonic beams having different steering angles with respect to the region of interest, and
wherein the sound speed calculator calculates the local sound speeds inside the region of interest by using one of the plurality of reception data for measuring a sound speed which has a wave front with the least disturbance.

4. The ultrasound diagnostic apparatus according to claim 2,
wherein the controller controls the transmission circuit and the reception circuit to obtain a plurality of reception data for measuring a sound speed by transmitting and receiving a plurality of ultrasonic beams having different steering angles with respect to the region of interest, and
wherein the sound speed calculator calculates the local sound speeds inside the region of interest by using one of the plurality of reception data for measuring a sound speed which has a wave front with the least disturbance.

5. The ultrasound diagnostic apparatus according to claim 1,
wherein the region-of-interest setting unit sets a plurality of regions of interest, and
wherein the sound speed calculator calculates local sound speeds in the regions of interest based on the obtained reception data for measuring a sound speed and uses calculated local sound speeds inside the regions of interest to interpolate a local sound speed in a region between the regions of interest.

6. The ultrasound diagnostic apparatus according to claim 2,
wherein the region-of-interest setting unit sets a plurality of regions of interest, and
wherein the sound speed calculator calculates local sound speeds in the regions of interest based on the obtained reception data for measuring a sound speed and uses calculated local sound speeds inside the regions of interest to interpolate a local sound speed in a region between the regions of interest.

7. The ultrasound diagnostic apparatus according to claim 3,
wherein the region-of-interest setting unit sets a plurality of regions of interest, and
wherein the sound speed calculator calculates local sound speeds in the regions of interest based on the obtained reception data for measuring a sound speed and uses calculated local sound speeds inside the regions of interest to interpolate a local sound speed in a region between the regions of interest.

8. An ultrasound image producing method, comprising the steps of:
transmitting an ultrasonic beam from a transducer array toward a subject;
producing reception data based on reception signals outputted from the transducer array having received ultrasonic echoes from the subject;
producing a B mode image based on the obtained reception data;
setting a region of interest in the produced B mode image;
detecting an abdominal wall of the subject in the B mode image;
setting a plurality of points in and near the region of interest;
transmitting and receiving ultrasonic beams emitted from the transducer array and so steered as to enter the abdominal wall vertically with forming transmission focuses at the points to obtain reception data for measuring a sound speed; and
calculating local sound speeds in the region of interest based on the obtained reception data for measuring a sound speed.
